(19) United States
(12) Patent Application Publication
(10) Pub. No.: US 2005/0014751 A1
(43) Pub. Date: Jan. 20, 2005

(54) Use of n-acyl homoserine lactones for the treatment of insulitis

(76) Inventor: David Idris Pritchard, Nottingham (GB)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/489,797

(22) PCT Filed: Sep. 13, 2002
(86) PCT No.: PCT/GB02/04191

(30) Foreign Application Priority Data
Sep. 13, 2001 (GB) 0122026.8

(51) Int. Cl. C07D 413/02; C 07D 4 5/02; A61K 31/5377; A61K 31/496; A61K 31/365

(52) U.S. Cl. 514/235.2; 514/254.1; 514/471; 544/152; 544/374; 549/321

(57) ABSTRACT

An immune response modulatory compound is described. The compound has been shown to inhibit lymphocyte proliferation and to down-regulate TNF-secretion by monocytes and/or macrophages with the consequent activation of Th 1 lymphocytes in humans or other animals.

The invention relates to N-acyl homoserine lactones which have immunosuppressant properties and to pharmaceutical compositions containing them.

Immunosuppressant compounds induce an inhibition of the immune response system. Compounds which are known to exhibit immunosuppressant activity include the fungal metabolite Cyclosporin A and the macrolide antibiotic (a metabolite from Streptomyces tsukabaensis) termed FK506. Both of these agents have been used clinically and experimentally to suppress the immune system in transplantation and in the treatment of a number of diseases.

Autoimmune diseases are disorders where the host discrimination of “self” versus “non-self” breaks down and the individual's immune system (both acquired and innate components) attacks self tissues. These diseases range from common entities such as rheumatoid arthritis, thyroid autoimmune disease and type 1 diabetes mellitus to less common entities such as multiple sclerosis and to rarer disorders such as myasthenia gravis. Advances in basic biomedical science and, in particular, in immunology have indicated that the main and fundamental lesion responsible for the induction and persistence of most autoimmune diseases resides within auto-reactive proliferating T lymphocytes. In fact, the majority of autoimmune diseases are linked to a loss of T cell homeostasis. The healthy immune system is held in balanced equilibrium, apparently by the contra-suppressive production of cytokines by T helper 1 (Th1) and T helper 2 (Th2) lymphocyte subsets. In autoimmunity, Th1 cytokines predominate; in allergy, Th2 cytokines take their place. A cytokine intimately associated with the development of Th1 biased responses and, consequently, autoimmune disease is TNF-α.

Both Cyclosporin A and FK506 have been used clinically in the treatment of autoimmune diseases with encouraging results.

The currently available immunosuppressant drugs have the disadvantage of a narrow therapeutic index, i.e., toxicity versus clinical benefit. The compounds are known to be nephrotoxic, neurotoxic and potentially diabetogenic and this has limited their use in the fields mentioned above. Problems also exist with the administration of these compounds, their bioavailability and the monitoring of their levels both clinically and in the laboratory.

We disclosed, in WO-A-95/01175, a class of compounds which exhibit antiallergic activity and inhibit the release of histamine, having the generic formula 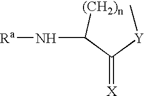
where: n is 2 or 3; Y is O, S or NH; X is O, S or NH; and R^{a} is C₁-C₁₈ alkyl or acyl which may be substituted.

Some of these compounds, and methods for their preparation, were previously disclosed in WO-A-92/18614 although that document discloses only that the compounds act as autoinducers and as agents for the control of gene expression. Compounds in the same series are also mentioned in Journal of Bacteriology, volume 175, number 12, June 1993, pages 3856 to 3862 but again there is no teaching that they might have any effect outside micro-organisms.

PCT/GB01/01435 describes the use of homoserine lactone compounds for topical application for autoimmune diseases such as psoriasis. The preferred active compound is N-(3-oxododecanoyl)-homoserine lactone. The active compound is preferably formulated in an ointment, cream or lotion.

G. Papaccio, Diabetes Res. Clin. Pract. vol.13, no.1, 1991, pages 95-102 discloses the use of N-acetylhomocysteine thiolactone as an enhancer of superoxide dismutase in an attempt to increase protection against chemically induced diabetes.

The use of N-acetylhomocysteine thiolactone to modify the IgE molecule is taught by J. Ljaljevic et al in Od. Med. Nauka, vol.24, 1971, pages 137-143 and Chemical Abstracts, vol.78, no.7, February 1973, abstract no. 41213a.

However, there is no suggestion in this paper of immunosuppression or of the inhibition of histamine release.

U.S. Pat. No. 5,591,872 discloses the compound N-(3-oxododecanoyl) homoserine lactone as an autoinducer molecule. In “Infection and Immunity”, vol.66, no.1, January 1998, the authors report the action of N-(3-oxododecanoyl)homoserine lactone (OdDHL) in inhibiting the concanavalin A mitogen stimulated proliferation of murine spleen cells and TNF-α production by LPS-stimulated adherent murine peritoneal macrophages.

We have now discovered a subclass of N-acyl homoserine lactones that exhibits an immunosuppressant activity greater than that exhibited by similar compounds outside of this subclass.

According to one aspect, the present invention provides a compound of the formula I 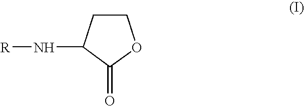
in which R is an acyl group of the formula II 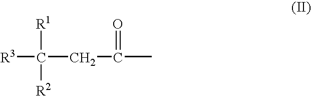
wherein one of R¹ and R² is H and the other is selected from OR⁴, SR⁴ and NHR⁴, wherein R⁴ is H or 1-6C alkyl, or R¹ and R² together with the carbon atom to which they are joined form a keto group, and R³ is a straight or branched chain, saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of R⁵ and R⁶ is selected from H and 1-6C alkyl or R⁵ and R⁶ together with the N atom form a morpholino or piperazino group, or any enantiomer thereof, with the proviso that R is not a 3-oxododecanoyl group.

The compounds of the present invention are capable of modulating the immune response in the living animal body, including human. In particular, they have an inhibitory effect on lymphocyte proliferation in humans and down-regulate TNF-α secretion by monocytes/macrophages and, in consequence, the activation of Th1 lymphocytes in humans. The present invention, therefore, provides a pharmaceutical composition comprising a therapeutically-effective amount of a compound of the invention as described herein, including an enantiomer thereof, together with a pharmaceutically-acceptable carrier or diluent.

A further aspect of the invention provides the use of a compound of the invention, including an enantiomer thereof, for the manufacture of a medicament for the treatment of a disease of a living animal body including human which disease is responsive to the activity of an immunosuppressant, for example an autoimmune disease. A yet further aspect of the invention relates to a method of treating a disease of a living animal body, including a human, which disease is responsive to the activity of an immunosuppressant, e.g., an autoimmune disease, which method comprises administering to the living animal body, including human, a therapeutically-effective amount of a compound according to the invention, as described herein including an enantiomer thereof.

Compounds of the invention have the general formula I given above. The group R in the formula I has the formula II

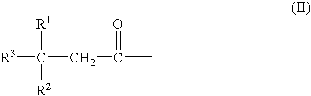

In formula II according to a first preferred embodiment one of R¹ and R² is H and the other is selected from OR⁴, SR⁴ and NHR⁴, in which R⁴ is H or a 1-6C alkyl group. Preferably, R⁴ is H. Such a definition of R¹ and R² gives rise to chirality at the carbon atom to which R¹ and R² are attached (C-3). The compounds of the invention can, thus, be in the form of racemates, optically active isomers or mixtures thereof. According to a particular preferred embodiment one of R¹ and R² is H and the other is OH.

According to this first preferred embodiment the group R³ in formula II is a straight or branched chain 8 to 11C aliphatic hydrocarbyl group which is saturated or which may be ethylenically unsaturated. The group may, further, be substituted by one or more substituent groups selected from halo, for example F, Cl, Br or I; 1-6C alkoxy, for example methoxy, ethoxy, n-propoxy, iso-propoxy, n-butoxy, iso-butoxy and tert-butoxy; carboxy including salts thereof, 1-6C alkoxycarbonyl, for example methoxycarbonyl, carbamoyl, for example N,N-dimethylcarbamoyl and NR⁵R⁶, wherein R⁵ and R⁶ are each selected from H and 1-6C alkyl or R⁵ and R⁶ together with the nitrogen atom to which they are attached form a morpholino group or a piperazino ring, optionally substituted at the 4-N by a methyl group. A particularly preferred R³ group in formula II above is a straight chain or branched chain 8 to 11C alkyl group which is optionally substituted by one substituent selected from Br, carboxy including salts thereof, and methoxycarbonyl. The substituent is typically, though not necessarily, attached in a terminal position on the alkyl group. Alternatively, the R³ group is a straight chain or branched chain 8-11C alkenyl group, preferably monoethenically unsaturated, which may be substituted by a substituent selected from Br, carboxy including a salt thereof, and methoxycarbonyl. Again, the substituent is typically, though not necessarily, attached in a terminal position on the alkenyl group.

In formula II above according to a second preferred embodiment the groups R¹ and R² together form an oxo group (═O) such that a keto group exists at the C-3 position in the acyl group. In such a case the group R³ in formula II will typically be:
(a) an optionally-substituted, saturated or ethylenically-unsaturated, straight or branched chain 8C aliphatic hydrocarbyl group;
(b) a substituted, saturated, straight or branched chain 9C aliphatic hydrocarbyl group;
   10-methyl-3-oxoundecanoyl;
   6-methyl-3-oxoundecanoyl;
   3-hydroxydodecanoyl;
   12-bromo-3-oxododecanoyl;
   3-oxotridecanoyl;
   13-bromo-3-oxododecanoyl;
   3-hydroxytetradecanoyl;
   3-oxotetradecanoyl;
   14-bromo-3-oxotetradecanoyl; and
   13-methoxycarbonyl-3-oxotridecanoyl.

Examples of acyl groups R of formula II above in which R³ is an ethylenically unsaturated hydrocarbyl group include:
3-oxo-12-tridecenoyl;
3-oxo-7-tetradecenoyl;
3-hydroxy-7-tetradecenoyl;
3-oxo-9-tetradecenoyl;
3-hydroxy-9-tetradecenoyl;
3-oxo-10-tetradecenoyl;
3-hydroxy-10-tetradecenoyl;
3-oxo-11-tetradecenoyl;
3-hydroxy-11-tetradecenoyl;
3-oxo-13-tetradecenoyl; and
3-hydroxy-13-tetradecenoyl.

The compounds of the present invention having the 3-oxo group may, in general, be prepared by a method comprising the steps of:
(1) reacting an acid having the general formula R³COOH, where R³ is as defined above, with Meldrum's acid (2,2-dimethyl-1,3-dioxane-4,6-dione) in the presence of 4-dimethylaminopyridine and N,N¹-dicyclohexylcarbodiimide in a dry organic solvent, such as dry dichloromethane, to give the acylated Meldrum's acid; and
(c) an optionally-substituted, ethylenically-unsaturated, straight or branched chain 9C aliphatic hydrocarbyl group;
(d) an optionally-substituted, saturated or ethylenically-unsaturated, straight or branched chain 10C aliphatic hydrocarbyl group; or
(e) an optionally-substituted, saturated or ethylenically-unsaturated, straight or branched chain 11C aliphatic hydrocarbyl group.

In the case where the group R³ is substituted, it will be substituted by one or more substituent groups selected from halo, for example F, Cl, Br or I; 1-6C alkoxy, for example methoxy, ethoxy, n-propoxy, iso-propoxy, n-butoxy, iso-butoxy and tert-butoxy; carboxy including salts thereof, 1-6C alkoxycarbonyl, for example methoxycarbonyl, carbamoyl, for example N,N-dimethylcarbamoyl, and NR⁵R⁶, wherein R⁵ and R⁶ are each selected from H and 1-6C alkyl or R⁵ and R⁶ together with the nitrogen atom to which they are attached form a morpholino group or a piperazino ring, optionally substituted at the 4-N by a methyl group.

According to one preferred embodiment the R³ group in formula II above is a straight chain or branched chain 8, 10 or 11C alkyl group which is optionally substituted by one substituent selected from Br, carboxy including salts thereof, and methoxycarbonyl. According to another preferred embodiment the R³ in formula II above is a straight chain or branched chain 9C alkyl group which is substituted by one substituent selected from Br, carboxy including salts thereof and methoxycarbonyl. The substituent is typically, though not necessarily, attached in a terminal position on the alkyl group.

According to yet another preferred embodiment the R³ group is a straight chain or branched chain 8-11C alkenyl group, preferably monoethenically unsaturated, which may be substituted by a substituent selected from Br, carboxy including a salt thereof, and methoxycarbonyl. The substituent is typically, though not necessarily, attached in a terminal position on the alkyl group.

Examples of acyl groups R of formula II above in which R³ is a saturated hydrocarbyl group include:
3-oxoundecanoyl;
11-bromo-3-oxoundecanoyl;
(2) reacting the acylated Meldrum's acid with L-homoserine lactone hydrochloride in an organic solvent, e.g., acetonitrile, to give the N-(3-oxoacylated)-L-homoserine lactone.

Where the appropriate acid is not available it may be prepared by, for instance, oxidising the appropriate alcohol using chromic acid.

The N-(3-hydroxyacyl)-L-homoserine lactone may be prepared by reducing the corresponding N-(3-oxoacyl)-L-homoserine lactone using sodium cyanoborohydride in acid conditions.

As mentioned above, the compounds of the present invention have use as pharmaceutically active ingredients in the treatment of an animal body, including the human body, suffering from a disease or disorder which is responsive to the activity of an immunosuppressant, particularly for the treatment of type I diabetes mellitus (type 1A autoimmune). The dosage administered to the animal body in need of therapy will, of course, depend on the actual active compound used, the mode of treatment and the type of treatment desired as well as on the body mass. The active compound may, of course, be administered on its own or in the form of an appropriate medicinal composition containing, for instance, an appropriate pharmaceutical carrier or diluent. Other substances can, of course, also be employed in such medicinal compositions, such as antioxidants and stabilisers, the use of which is well known to persons skilled in the art.

Preferably, the compound is orally administered. The present inventors have found that, in the treatment of insulitis or type I diabetes mellitus, at present in NOD (non-obese diabetic) mice, that the compounds of the present invention have greater efficacy and bioavailability than the conventionally used compound vehicle DMSO. Furthermore, the compounds of the present invention perform at least comparably to accepted immune modulators such as CsA or antibodies to TNF without exerting overt immune toxicity.

In comparative in vitro studies for the prevention of cell proliferation CSA and dexamethasone performed better than OdDHL. However, unexpectedly, when the assay was performed in vivo OdDHL performed better than CSA and dexamethasone (see below), leading the present inventors to conclude that in an in vivo situation CSA and dexamethasone are either inactivated or prevented from acting by metabolic processes or by physiological breakdown of the compound whereas OdDHL is either resistant to these processes or changes or is somehow further activated by them. From this it is believed that a similar result will be achieved for insulitis.

The most preferred compound of the present invention is OdDHL[N-(3-oxododecanoyl)-L-homoserine lactone] and derivatives or substituents thereof, as set out in PCT/GB01/01453, the content of which is incorporated herein by reference.

In another aspect of the invention, the OdDHL or the other related immune modulatory compounds may be used in the identification of molecular targets and novel immunophilins in cells, preferably pancreatic beta cells or autoreactive leucocytes by constructing affinity matrices incorporating the compounds.

Two possible chemical strategies which may be employed in the production of such an affinity matrix are the synthesis of side-chain functionalised, for example C2 or C4-functionalised OdDHL (3O, C12-HSL) derivates (X=(CH₂)ₙCOOH) as shown for side chains in Formulae I and II below. The length of the alkyl chain (n=1, 2, 3 etc) will determine the efficiency of binding to the novel immunophilins.

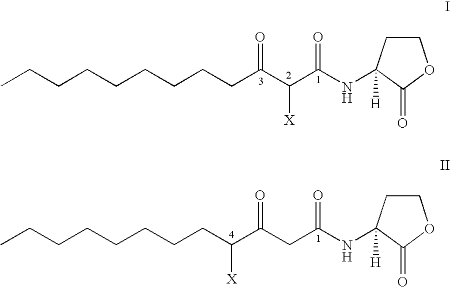

In the above formulae, X may be selected from Br, Cl, I or (CH₂)nCOOH.

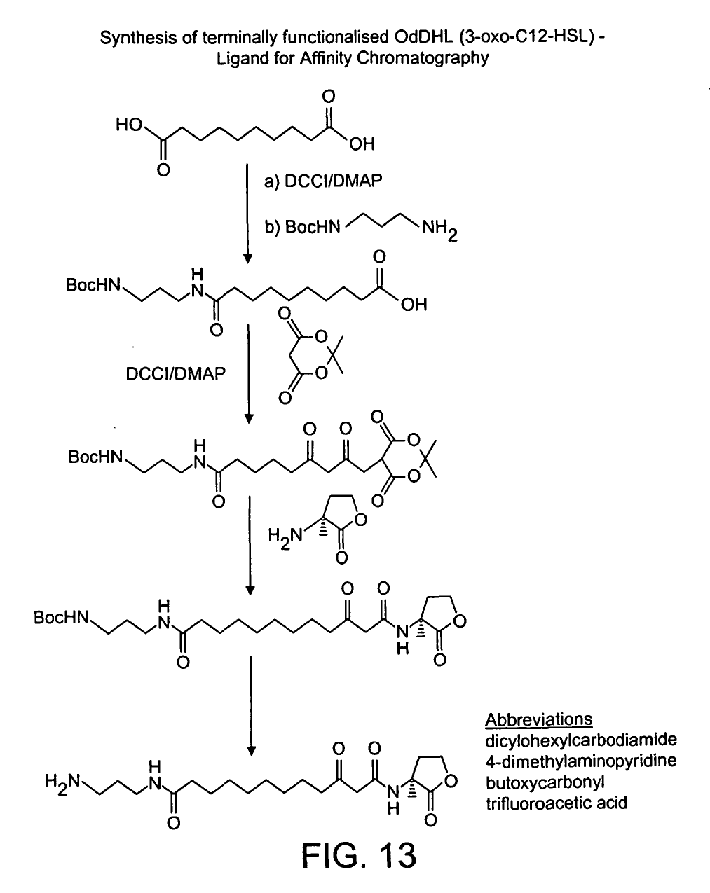

Alternatively, the molecule may be terminally functionalised following the schemes shown in FIGS. 13 and 14.

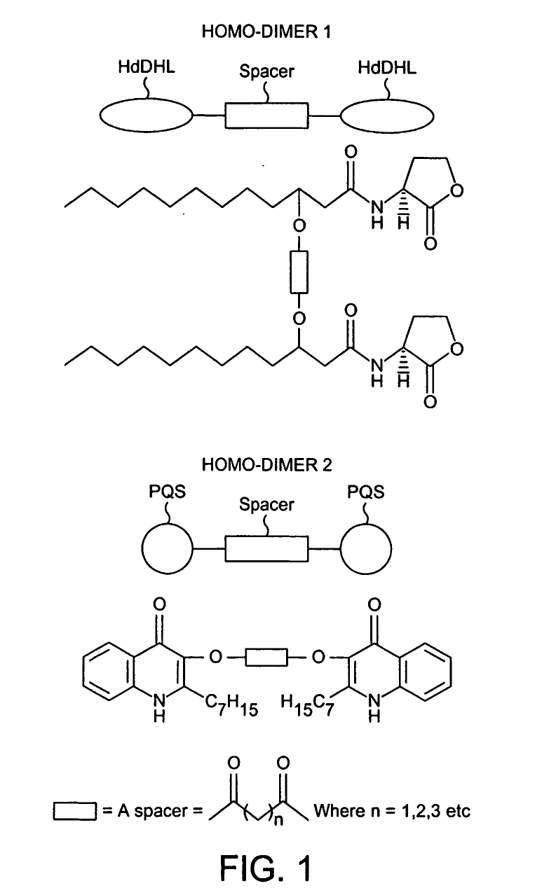

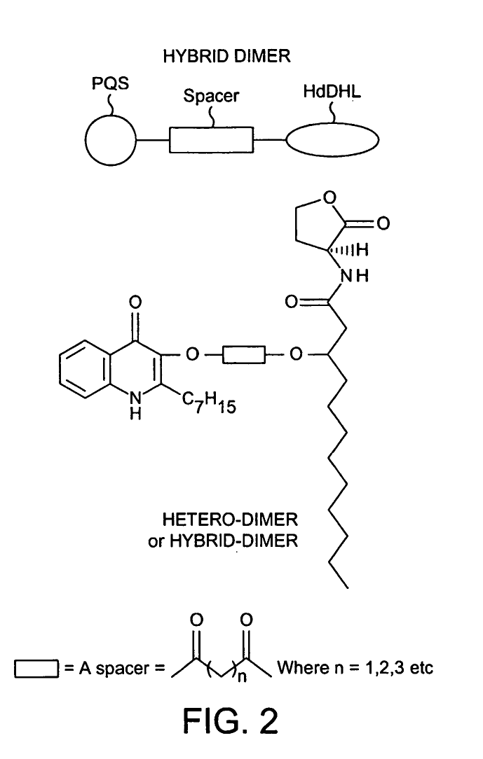

In a further aspect of the invention, more potent immune modulatory agents can be generated by the synthesis of bivalent OdDHL, PQS and hybrids. These bivalent ligands can be constructed by, linking PQS and HdDHL (3OH, C12-HSL) through a spacer of optimum length to provide a Homo-dimer (See FIG. 1) or Hetero-dimer (See FIG. 2).

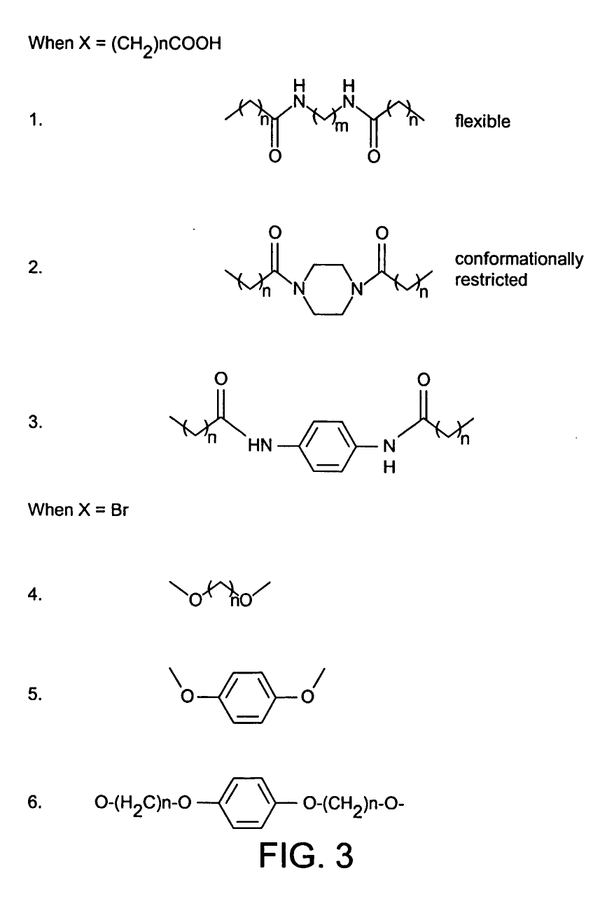

Preferably, the two molecules will be linked though their respective 3-OH substituents via a spacer. Alternatively, for the Homo-dimers, when the C2 or C4 substituent is carboxyalkyl [(CH₂₎ₙCOOH], OdDHL-dimers can be synthesised by the method described in J.Med. Chem. 2001, 44, 1615-1622. Without wising to be bound by theory, the present inventors believe that the nature of the substituent at C2 or C4, that is the halogen or the carboxyalkyl will determine or limit the nature of the spacer linking the two molecules. For example, the choice of the spacer is likely to have an effect on the immunomodulatory properties of the dimer through possible sites of the linkage of the spacer and the nature of the covalent linkage. Additionally, the length of the spacer is important when determining the effectiveness of the cross-linking since it is desired to create a compound which exhibits a potency that is greater than that derived from the sum of its two monovalent pharmacophores. It is desirable to use a flexible or conformationally restricted spacer, especially in order to prevent adverse effect with steric hindrance. Some examples of spacers are shown in FIG. 3.

Optionally, the carboxyalkyl substituent at C2 or C4 can be tagged (for example, with a colorimetric or fluorometric tag) for cell compartmentalisation assays or targeting studies, or simply for use in the affinity matrix studies, for example to show binding. This strategy of tagging via C2 or C4 is especially preferred as the biologically important parts of the molecule are unaffected or at least are still available for chemical, biochemical or physiological interactions.

Embodiments of the invention will now be described in more detail, by way of example only, with reference to the following examples which are illustrated with reference to the accompanying drawings, of which

FIGS. 1, 2 and 3 have already been described;

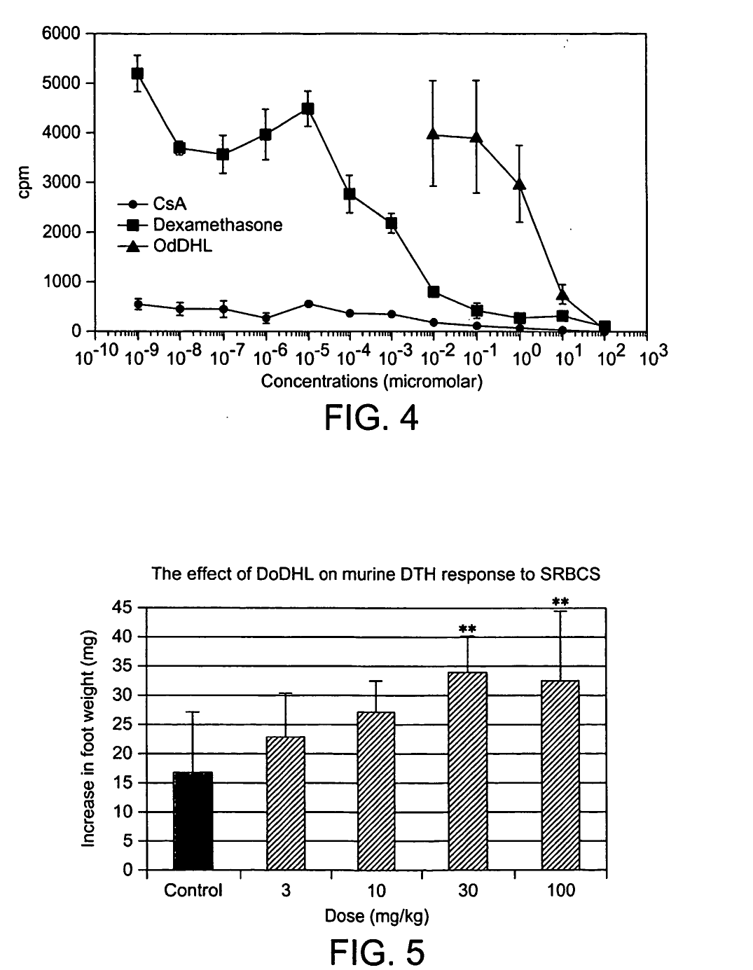

FIG. 4 is a graph showing the comparative in vitro anti proliferative effects of CSA, dexamethasone and OdDHL;

FIG. 5 is a graph showing the influence of oral OdDHL on a murine in vivo DTH response to SRBCs;

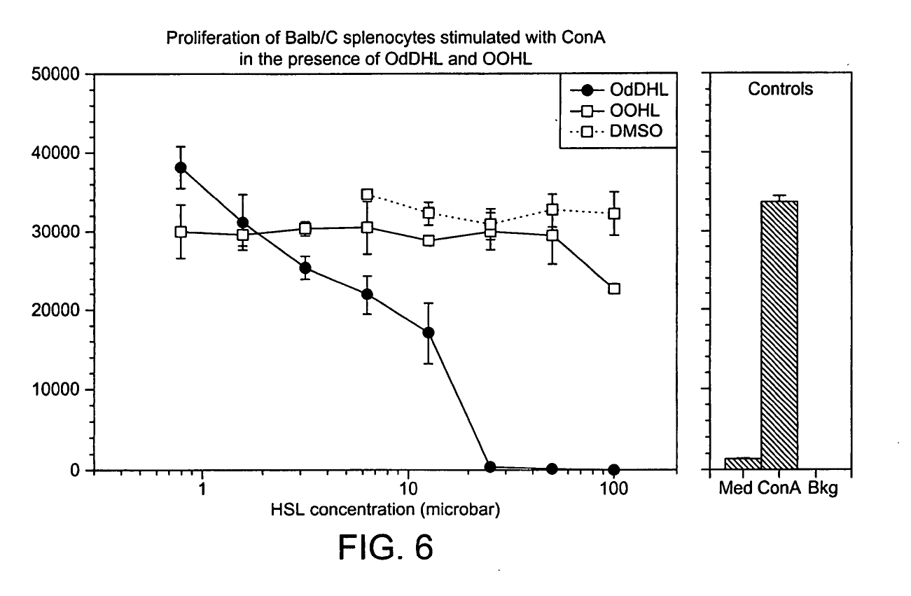

FIG. 6 is a graph showing the proliferation of Balb/C splenocytes stimulated with ConA. in the presence of OdDHL and OOHL;

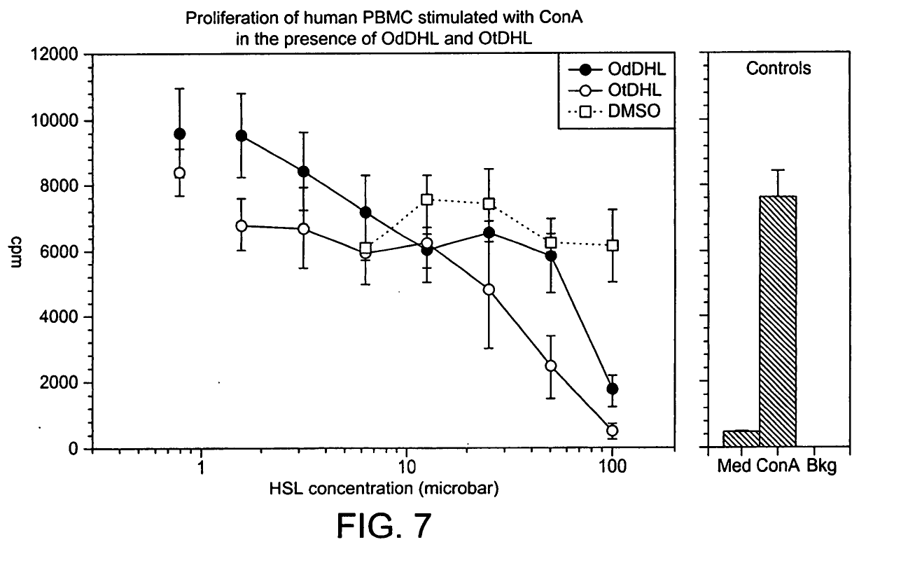

FIG. 7 is a graph showing the proliferation of human PBMC stimulated with ConA in the presence of OdDHL and OtDHL;

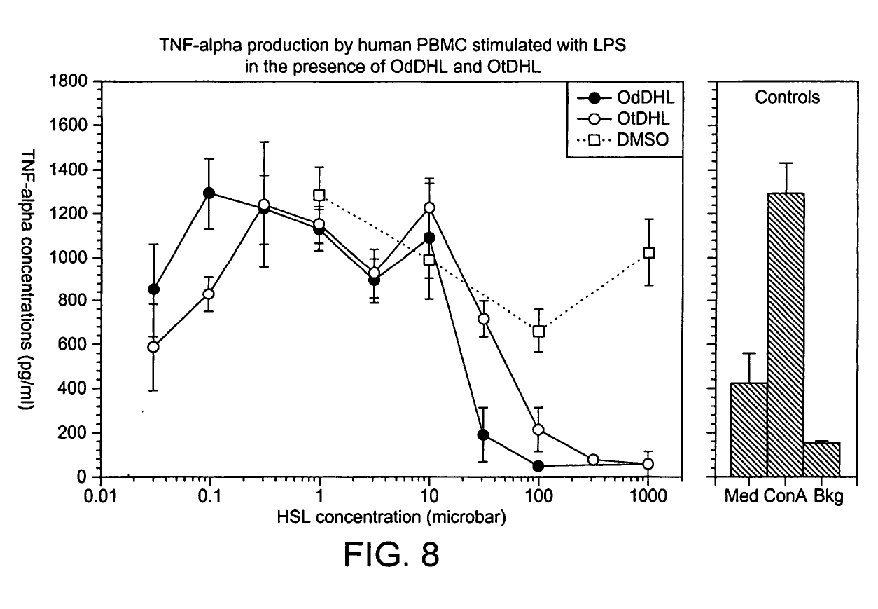

FIG. 8 is a graph showing TNFα production by human PBMC stimulated with LPS in the presence of OdDHL and OtDHL;

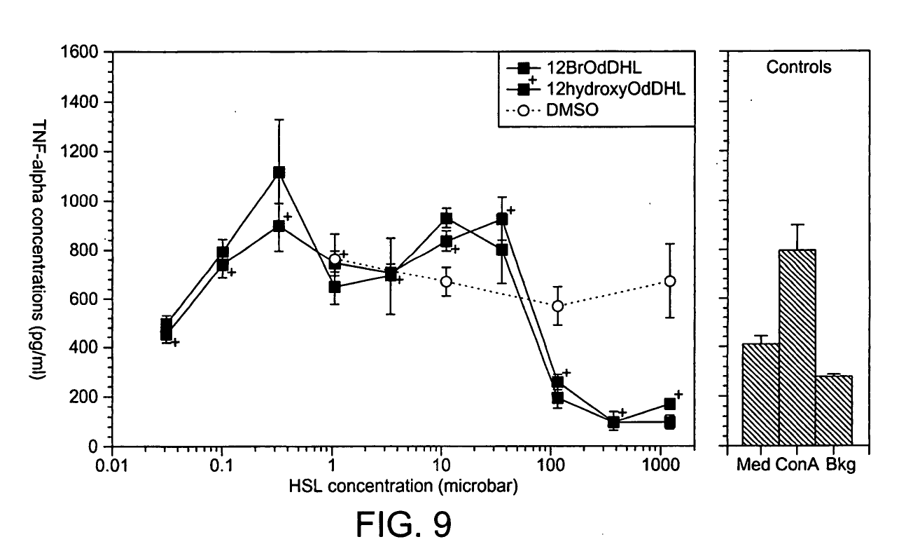

FIG. 9 is a graph showing TNF-α production in the presence of test compounds;

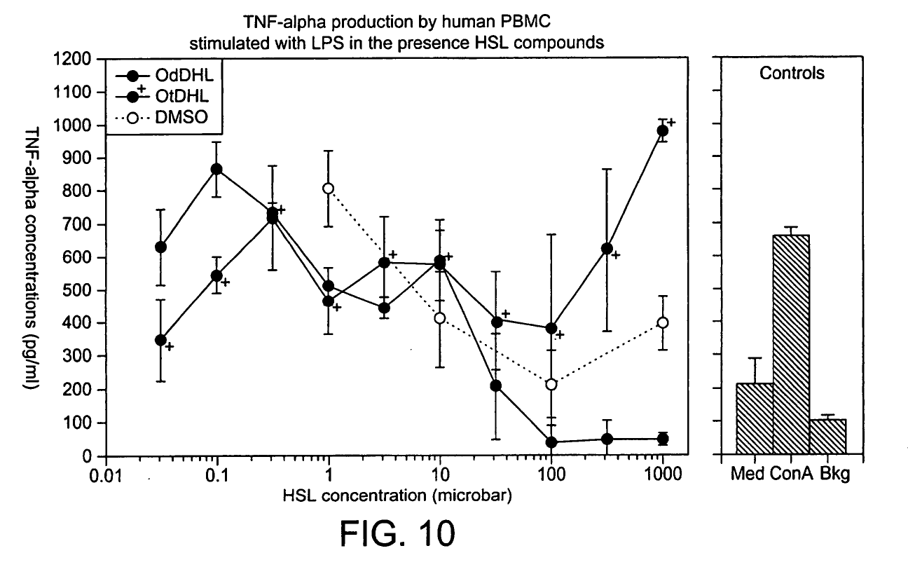

FIG. 10 is a graph showing TNF-α production by human PBMC in the presence of test compounds;

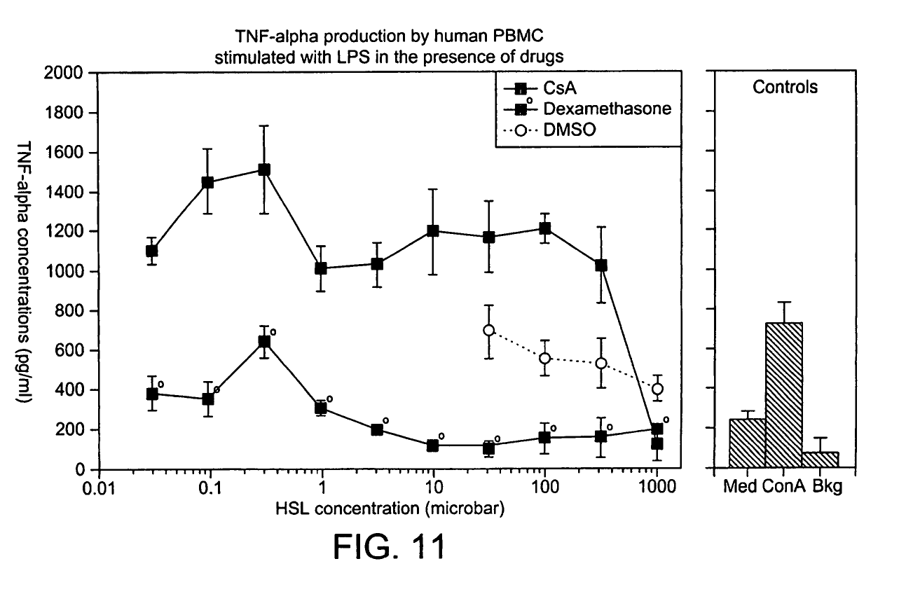

FIG. 11 is a graph showing TNFα production in the presence of drugs;

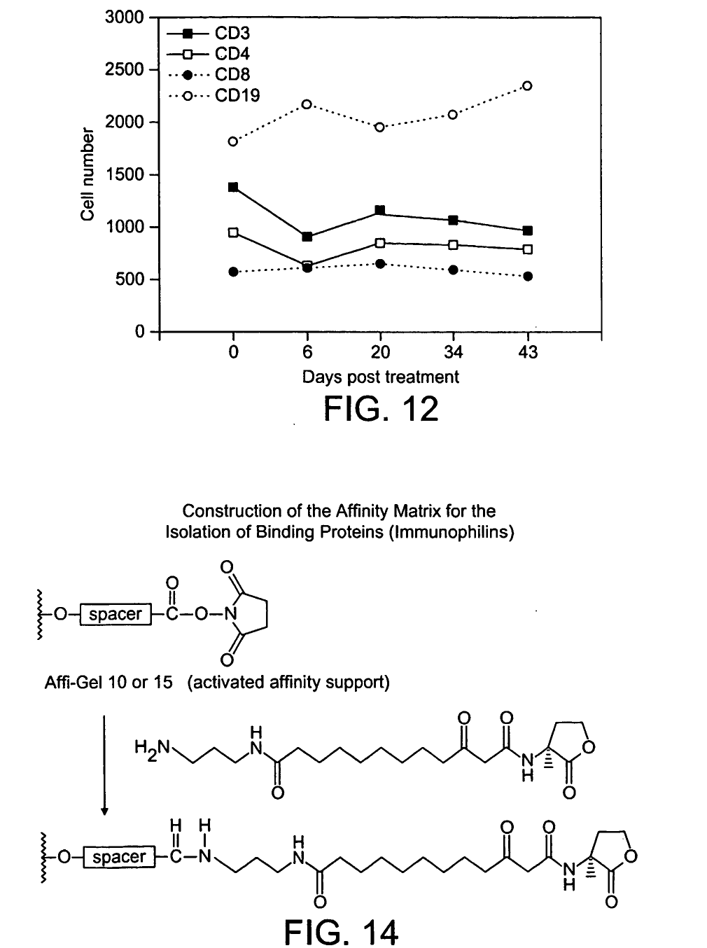

FIG. 12 is a graph showing lack of overt toxicity of OdDHL.

FIG. 13 shows schematically the synthesis of a terminally functionalised OdDHL; and

FIG. 14 shows schematically the synthesis of a terminally functionalised OdDHL.

### EXAMPLES

### Example 1

N-(3-oxododecanoyl)-L-homoserine Lactone (OdDHL)

To a solution of decanoic acid (2 mmol) in dry dichloromethane (20 ml) was added 4-dimethylaminopyridine (2.1 mmol), N,N′-dicyclohexylcarbodiimide (2.2 mmol) and Meldrum's acid (2 mmol). The solution was stirred at room temperature overnight and then filtered to remove the precipitated dicyclohexylurea. The filtrate was evaporated to dryness and the residue redissolved in ethyl acetate. The ethyl acetate solution was washed with 2 M hydrochloric acid, dried over anhydrous magnesium sulphate and concentrated to afford the decanoyl Meldrum's acid.

To a stirred solution of the decanoyl Meldrum's acid (1 mmol) in acetonitrile (30 ml) was added L-homoserine lactone hydrochloride (1 mmol) and triethylamine (1.2 mmol). The mixture was stirred for 2 hours and then refluxed for a further 3 hours. The solvent was removed by rotary evaporation to give a residue that was redissolved in ethyl acetate. The organic solution was sequentially washed with saturated sodium hydrogen carbonate solution, 1M potassium hydrogen sulphate solution and saturated sodium chloride solution. After drying over anhydrous magnesium sulphate, the organic extract was evaporated to dryness and the residue purified by preparative layer chromatography on silica plates.

¹H NMR (250 MHz, CDCl₃) δ 0.9 (3H, t, CH₃), 1.27 (12H, m, CH₃(CH₂)₆), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 4.27 (1H, m, 5α-H), 4.48 (1H, td, 5β-H), 4.58 (1H, m, 3-H), 7.64 (1H, d, NH).

The procedure described above in Example 1 was followed to prepare other N-(3-oxoacylated)-L-homoserine lactones as described below using, in each case, the appropriate carboxylic acid.

### Example 2

N-(12-Bromo-3-oxododecanoyl)-L-homoserine Lactone (12BrOdDHL)

¹H NMR (250 MHz, CDCl₃) δ 1.27 (10H, m, BrCH₂CH₂(CH₂)₅), 1.45 (2H, m, BrCH₂CH₂CH₂), 1.59 (2H, m, CH₂CH₂CO), 2.22 (1H, m, 4α-H), 2.52 (2H, t, CH₂CO), 2.76 (1H, m, 4β-H), 3.47 (2H, s, COCH₂CO), 3.53 (2H, t, BrCH₂), 4.27 (1H, m, 5α-H), 4.48 (1H, td, 5β-H), 4.58 (1H, m, 3-H), 7.64 (1H, d, NH).

### Example 3

N-(12-Hydroxy-3-oxododecanoyl)-L-homoserine Lactone (12OHOdDHL)

Using 10-acetoxydecanoic acid in the general procedure as described above in Example 1 afforded the N-(12-acetoxy-3-oxododecanoyl)-L-homoserine lactone. The latter when refluxed in 1M hydrochloric acid, yielded the title product.

¹H NMR (250 MHz, CDCl₃) δ 1.27 (12H, m, HOCH₂(CH₂)₆), 1.59 (2H, m, CH₂CH₂CO), 1.89 (1H, t, OH), 2.22 (1H, m, 4α-H), 2.52 (2H, t, CH₂CO), 2.76 (1H, m, 4β-H), 3.47 (2H, s, COCH₂CO), 3.60 (2H, t, HOCH₂), 4.27 (1H, m, 5α-H), 4.48 (1H, td, 5β-H), 4.58 (1H, m, 3-H), 7.64 (1H, d, NH).

### Example 4

Synthesis of a Homo and a Hybrid-dimer by Linking through 3-OH Substituents

N,N′-Dicyclohexylcarbodiimide (DCCl) and 4-dimethylaminopyridine (DMAP) catalysed acylation of the 3-OH substituent of the PQS with glutaric acid mono t-butyl ester would furnish the acylated PQS (Scheme 1). Removal of the tBu protection by acidolysis is followed by esterification of the resultant carboxylic acid either with the 3-OH substituent of another molecule of PQS to furnish the homo-dimer or HdDHL (3-OH, C12-HSL) to deliver the desired hetero-dimer.

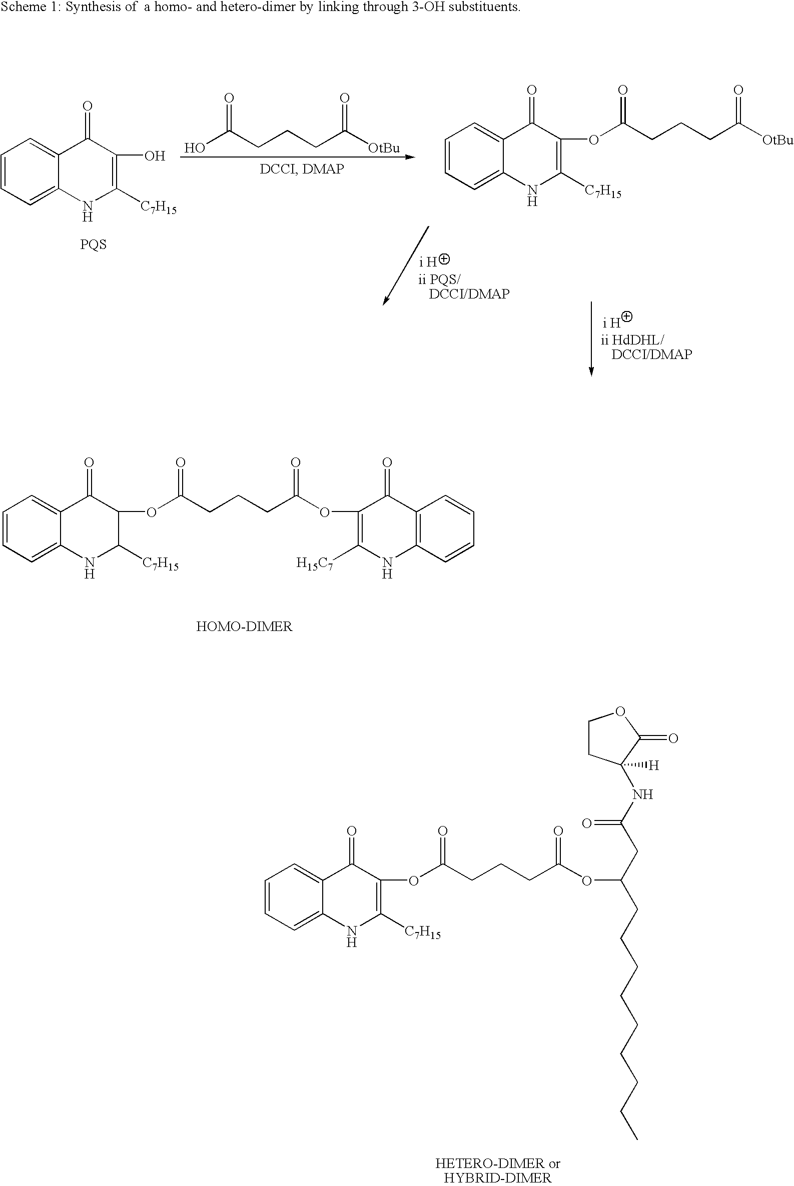

### Example 5

Effect of OdDHL on Insulitis

To investigate the effect of OdDHL on insulitis or type I diabetes mellitus, mice (NOD) with a genetic predisposition to develop IDDM (insulin dependent diabetes mellitus) were treated with OdDHL.

The animals were dosed at 100 mg/kg intraperitoneally from 4 weeks of age, 3 times a week for four weeks. Insulitis was assessed at 14 weeks.

In the table a score of 3 or more (DMSO and PBS) represents severe grade insulitis and 1.3 (OdDHL) represents little infiltration (a score of 1 would represent low grade peri-insulitis).

The results for 100 mg/kg are shown in Table 1 below. The mice were scored according to the method of Beales et al (European Journal of Pharmacology 357(1998) 221-225).

TABLE 1

| | DMSO | PBS | OdDHL |
|---|---|---|---|
| Animal Number | Slide Score | Slide Score | Slide Score |
| 1 | 3.160 | | |
| 2 | | | 1.000 |
| 3 | | | 1.418 |
| 4 | | 3.372 | |
| 5 | | 1.891 | |
| 6 | 3.855 | | |
| 7 | 4.352 | | |
| 8 | | 3.937 | |
| 9 | | | 1.936 |
| 10 | 3.202 | | |
| 11 | | | 1.478 |
| 12 | | 2.690 | |
| 13 | | 3.806 | |
| 14 | 1.735 | | |
| 15 | | 3.184 | |
| 16 | | | 1.154 |
| 17 | 2.019 | | |
| Group Total | 18.23 | 18.880 | 6.986 |
| Group Mean | 3.054 | 3.140 | 1.397 |
| Standard | 1.01691 | 0.76115 | 0.35853 |
| Derivation | | | |

### Example 6

Oral Availability of OdDHL

To show that OdDHL is orally available, the anti-sheep red blood cell (SRBC) responses of mice were measured in accordance with the following experiment. Eight week old, female Balb/c mice were kept on a 12 hour light/dark cycle and fed food and water ad libitum. SRBCs in Alseviers solution (TCS Biologicals, SB069) were washed and centrifuged (800×g for 20 minutes at room temperature) three times with 0.9% NaCl in distilled water. Cells were counted using a haemocytometer. On day 0 animals were immunised intraperitoneally (IP) with 5×10⁶ (low dose) or 5×10⁸ (high dose) SRBCs in saline. On day 5, animals were challenged with 20 μl SRBCs at 5×10⁹/ml in the left hind paw. The contralateral paw was injected with 20 μl saline alone. After 24 hours the animals were sacrificed with a rising concentration of CO₂ and bled by cardiac puncture. The hind feet were severed at the ankle joint and weighed.

Animals were dosed po from day 0 to day 5 with OdDHL. OdDHL was suspended in 0.25% (wt/vol) cellosize (Boots Co. PLC, Nottingham UK) in distilled water containing 1.5% (vol/vol) Tween 80 (Registered Trade Mark, Sigma) for po dosing at 3, 10, 30 and 100 mg/kg. Animals received 0.1 ml po.

It was found that immunisation of Balb/c mice with 5×10⁶ SRBCs evokes a delayed type hypersensitivity response (type IV) when challenged with SRBCs in the footpad. This manifests itself as an inflammatory response causing an increase in the size of the foot. Very little antibody is produced. OdDHL at 3, 10, 30 and 100 mg/kg caused a dose dependent increase in footweight of 37%, 60%, 100% and 93% respectively (see FIG. 5). Mice dosed with OdDHL had higher footweights when compared to vehicle dosed animals. The 30 and 100 mg/kg doses (** on graph) are significant as determined by Dunnett's multiple comparison's test after one-way analysis of variance (p<0.01). This assay is considered by the pharmaceutical industry to be Th2 dependent, indicating the effect of compound treatment on contra-regulating T-helper 1 lymphocytes.

### Example 7

Immunomodulatory Activity of Homoserine Lactone Compounds

Materials and Methods

I. ConA Mitogen-stimulated Proliferation of Murine Splenocytes

The concanavalin A (ConA) cell proliferation assay was used to assess the effect of homoserine lactone (HSL) compounds on T-cell activation and proliferation. Proliferation was assessed by the incorporation of [³H]-thymidine into DNA. Eight-week-old female BALB/c mice were obtained from Harlan (Bicester, Oxon, UK) and given food and water ad libitum. Splenocyte suspensions were prepared by removing the spleens and placing them into. RPMI 1640 medium. The spleens were forced through 70-μm-pore-size wire gauzes using the plunger from a 5-ml syringe to produce a single cell suspension. The cells were pelleted by centrifugation, and erythrocytes were lysed with 0.017M Tris, 0.144M ammonium chloride buffer, pH 7.2. Leucocytes were washed twice with RPMI 1640 medium with 2% (vol/vol) foetal calf serum (FCS) and resuspended in complete cell culture medium (CTCM) consisting of RPMI 1640 medium with 5% FCS, 2 mM L-glutamine, and 5×10⁻⁵ M 2-mercaptoethanol. HSL compounds were tested at doubling down dilutions ranging from 1 mM to 0.1 μM in a final volume of 200 μl of CTCM, containing ConA (Sigma, Poole, UK) at 1 μg/ml and 100,000 spleen cells. Following incubation for 48 h at 37° C. in 5% CO₂-air, 0.25 μCi [³H]-thymidine (Amersham) in 10 μl volume made up in RPMI 1640 medium was added and the cells were incubated for a further 24 h. Cells were harvested onto fibreglass filters with a Packard filtermate harvester. After the addition of 25 μl of MicroScint-O (Packard) to each well the filters were counted with the Packard TopCount scintillation counter.

Mitogen (Concanavalin A) induced murine splenocyte proliferation was indicated by the incorporation of tritated thymidine into the DNA in the mouse spleen cells as shown by counts per minute using the scintillation counter. The inhibitory effect of an HSL compound being tested on cell proliferation was indicated by a reduction in counts per minute. FIG. 6 shows the plots of counts per minute (cpm) against the concentrations (micromolar) of the HSL compounds N-(3-oxododecanoyl)-L-homoserine lactone (OdDHL) and N-(3-oxooctanoyl)-L-homoserine lactone (OOHL) and the vehicle dimethylsulphoxide (DMSO). It can be seen, from this figure, that OdDHL inhibits splenocyte proliferation. In contrast, OOHL and DMSO failed to inhibit proliferation.

The IC50 value, i.e., the concentration (micromolar) of a compound which inhibits cell proliferation thymidine incorporation by 50% was determined for several compounds of the present invention and these IC 50 values are shown in column A of the Table below.

II. ConA Mitogen-stimulated Proliferation of Human PBMC

Blood specimens were obtained with consent from healthy human volunteers. Human peripheral blood mononuclear cells (PBMC) were isolated from heparinised whole blood by buoyant density centrifugation over Histopaque 1077 (Sigma, Poole, UK) at 600 g for 20 minutes. PBMC harvested from the ‘buffy’ layers were washed twice with RPMI 1640 medium and resuspended in CTCM. HSL compounds were tested at similar dilutions as for murine splenocytes in 200 μl of CTCM, containing 1 μg/ml of ConA and 100,000 PBMC. Human PBMC were incubated for 48 h at 37° C. in 5% CO₂-air, followed by pulsing with 0.25 μCi [³H]-thymidine (see above). After a further incubation of 24 h cells were harvested onto fibreglass filters and then counted in the presence of MicroScint-O with the Packard TopCount.

Concanavalin induced cell proliferation of human peripheral blood mononuclear cells (PBMC) was tracked, as described in I above, by a measurement of counts per minute using the scintillation counter. The inhibitory effect of an HSL compound being tested on cell proliferation was indicated by a reduction in counts per minute. FIG. 7 shows the plots of cpm against the concentrations of OdDHL, N-(3-oxotetradecanoyl)-L-homoserine lactone (OtDHL) and DMSO (vehicle). As can be seen, both OdDHL and OtDHL inhibited proliferation of human PBMC stimulated with Concanavalin A.

The IC50 values for several HSL compounds of the invention were determined and these are shown in columns B, C and D in the Table below. Columns B, C and D represent different sources of human PBMC samples used.

III. TNF-alpha Production from LPS-stimulated Human PBMC

Bacterial lipopolysaccharide (LPS) stimulates the production of a variety of cytokines, including TNF-alpha, from human PBMC; these cytokines in turn influence the development of T cells, supporting a T helper 1 conducive milieu. Human PBMC prepared from whole blood by buoyant density centrifugation were resuspended in CTCM. HSL compounds were again tested at similar dilutions as for murine splenocytes in 200 μl of CTCM, containing 5×10⁻⁵ μg/ml LPS Escherichia coli strain 055:B5 (Sigma, Poole, UK) and 100,000 PBMC. Following incubation for 24 h at 37° C. in 5% CO₂-air, the cell culture supernatants were collected and tested for TNF-alpha levels by ‘sandwich’ ELISA. Briefly, 96-well Nunc MaxiSorp (Life Technologies, Paisley, UK) plates were coated with 50 μl of a 2 μg/ml solution of mouse anti-human TNF-alpha monoclonal antibody (Pharmingen, UK) in 0.05 M carbonate/bicarbonate buffer, pH 9.6 overnight at 4° C. After washing the plates three times with PBS-Tween, which contained phosphate buffered saline (PBS) with 0.5% (vol/vol) Tween 20 (Sigma, Poole, UK), the plates were blocked with 1% (wt/vol) bovine serum albumin (BSA) (Sigma, Poole, UK) at room temperature for 2 h. Following three washes with PBS-Tween, 50 μl of cell culture supernatants were added and incubated overnight at 4° C.; standard human TNF-alpha (Pharmingen, UK) ranging from 2000 to 31.25 pg/ml were included for each plate. After four washes with PBS-Tween, 50 μl of a second antibody, biotinylated mouse anti-human TNF-alpha monoclonal antibody (Pharmingen, UK) was added at 0.5 μg/ml diluted in 1% BSA in PBS-Tween and incubated at room temperature for 1 h. Following four washes, the bound biotinylated antibody was detected with 50 μl of a 1:1,000 dilution of Streptavidin-peroxidase (Pharmingen, UK). At the end of an hour incubation at room temperature, the plates were thoroughly washed six times with PBS-Tween and the assay was developed by the addition of 100 μl of 0.1 mg/ml of tetramethyl benzidine subtrate (Sigma, Poole, UK) in 0.1 M sodium acetate buffer, pH 6 containing 0.03% H₂O₂. The enzyme reaction was stopped with 50 μl of 2.5 M H₂SO₄ after an incubation of 10 minutes at room temperature and the development was read at 450 nm with a spectrophotometric 96-well plate reader (Dynex).

The effect of the concentration of certain HSL compounds of the invention on LPS induced TNF-α production by human PBMC was observed. FIG. 8 shows plots of TNF-α concentrations (pg/ml) against the concentration (micromolar) of OdDHL, OtDHL and DMSO (vehicle). As can be seen, both OdDHL and OtDHL inhibited the secretion of the T helper 1-supporting cytokine TNF-α. The IC50 values, i.e., the concentration (micromolar) of a compound which inhibits TNF-α secretion by 50%, was determined for some of the HSL compounds of the invention and these are shown in column E in the Table below.

Similar studies were carried out using, as the HSL compounds, N-(12-bromo-3-oxododecanoyl)-L-homoserine lactone (12BrOdDHL) and N-(12-hydroxy-3-oxododecanoyl)-L-homoserine lactone (12hydroxyOdDHL) and the plots for these are shown in FIG. 9. For comparison purposes, similar studies were carried out using, as the HSL, the known shorter side chain compound N-(3-oxohexanoyl)-L-homoserine lactone (OHHL) and the plot for this is shown in FIG. 10. The difference in activity between OHHL and OdDHL is marked. Also for comparison purposes, similar studies were carried out using the known drugs dexamethasone and Cyclosporin A (CsA) and the plots for these are shown in FIG. 6. The IC50 value for dexamethasone was determined to be 500.

IV. Optimisation of Cell Culture Conditions

In the cell culture assays the number of cells used (mouse splenocytes and human PBMC) was initially optimised to 100,000 cells per well. The optimal dose of ConA of 1 μg/ml used in the cell proliferation assays was determined from ConA titration curves. A similar titration curve was established for LPS stimulation to obtain an LPS concentration which stimulated a suboptimal level of TNF-alpha release from human PBMC.

### Example 8

Comparative Immune Toxicity Assessment

The overt immune toxicity of OdDHL was investigated using the dose regime described above in Example 5 for the alleviation of insulitis or diabetes in NOD mice. Treated mice gained weight identically to their non-treated littermates. The results are shown in FIG. 12. Splenocytes from the mice were taken along the time course of treatment (3 times a week for 4 weeks) and were stained with anti-CD-3 (pan T-cell), anti-CD4 (helper T-cell), anti-CD8 (cytotoxic T-cell) and anti-CD19 (B cell) antibodies for 30 minutes on ice. Cells were washed twice in PBS/BSA and then fixed in 0.5% formaldehyde. Cell phenotypes were analysed on BD FACScan. As can be seen from FIG. 12, a lack of overt immune toxicology was displayed during treatment with the immune cell populations remaining constant in proportion throughout the course of treatment.

### Example 9

In vivo Effects of CSA. Anti-TNFα and OdDHL

Dose regimes of Cyclosporin A, anti-TNFα antibody and OdDHL were compared in the diabetes model. NOD mice were dosed at 25 mg/kg on alternate days for 160 days following the methodology of Mori et al (Diabetologia (1986) 29: 244-247), the content of which is incorporated herein by reference, for the investigation of CSA, and Anti TNF at 12 mg per individual, 0.5 mg three times a week for 8 weeks in accordance with the method of Suk et al (J. Immunology (2001) 166: 4481-4489), the content of which is incorporated herein by reference, was used for TNFα. OdDHL was dosed at 30 mg/kg, 3 times per week for 4 weeks as above. When compared to the reported data of Mori and Suk (Supra) OdDHL displayed more effective in vivo action than in vitro contrary to what would be expected from the in vitro results previously discussed (page 8).

### Example 10

Confirmation of Alleviation of Diabetes in NOD Mice

The ability of the lead compound (OdDHL) to alleviate diabetes in NOD mice was determined by treating the mice 3 times per week for 4 weeks at 30 mg/kg as above. DMSO and OHHL were used as controls.

Diabetes was diagnosed using Uristix and a level of above 6 mMol glucose/I was taken to indicate the presence of diabetes. The results are shown in Table 2, which shows the incidence of diabetes at significant weeks of the experiment.

TABLE 2

| Week of Experiment | 20 | 30 |
|---|---|---|
| DMSO | 7/12 | 15/20 |
| OHHL | 7/10 | 12/20 |
| OdDHL | 0/11 | 5/20 |

A Kaplan-Meier analysis of cumulative incidence of diabetes shows:

| | |
|---|---|
| OdDHL vs DMSO | p = 0.0004 |
| OdDHL vs OHHL | p = 0.009 |
| DMSO vs OHHL | = non significant (NS) |

1. An immune response modulatory compound of the formula I 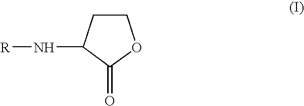
in which R is an acyl group of the formula II 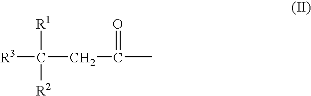
wherein one of R¹ and R² is H and the other is selected from OR⁴, SR⁴ and NHR⁴, wherein R⁴ is H or 1-6C alkyl, or R¹ and R² together with the carbon atom to which they are joined form a keto group, and R³ is a straight or branched chain, saturated or unsaturated aliphatic hydrocarbyl group containing from 8 to 11 carbon atoms and is optionally substituted by one or more substituent groups selected from halo, 1-6C alkoxy, carboxy, 1-6C alkoxycarbonyl, carbamoyl optionally mono- or disubstituted at the N atom by 1-6C alkyl and NR⁵R⁶ wherein each of R⁵ and R⁶ is selected from H and 1-6C alkyl or R⁵ and R⁶ together with the N atom form a morpholino or piperazino group, or any enantiomer thereof, with the proviso that R is not a 3-oxododecanoyl group.

2. A compound according to claim 1, in which group R in the formula I has the formula II 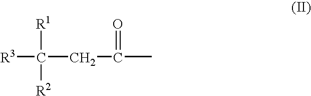
in which one of R¹ and R² is H and the other is selected from OR⁴, SR⁴ and NHR⁴, in which R⁴ is H or a 1-6C alkyl group.

3. A compound according to claim 2, in which R⁴ is H.

4. A compound according to claim 3, in which one of R¹ and R² is H and the other is OH.

5. A compound according to any one of claims 1 to 4, in which R³ of formula II is a straight or branched chain 8 to 11C aliphatic hydrocarbyl group which is saturated or ethylenically unsaturated.

6. A compound according to claim 5, in which the R³ group may be further substituted by one or more substituent groups selected from halo, F, Cl, Br or I; 1-6C alkoxy, methoxy, ethoxy, n-propoxy, iso-propoxy, n-butoxy, iso-butoxy and tert-butoxy; carboxy including salts thereof, 1-6C alkoxycarbonyl, methoxycarbonyl, carbamoyl, N,N-dimethylcarbamoyl and NR⁵R⁶, wherein R⁵ and R⁶ are each selected from H and 1-6C alkyl or R⁵ and R⁶ together with the nitrogen atom to which they are attached form a morpholino group or a piperazino ring, optionally substituted at the 4-N by a methyl group.

7. A compound according to claim 5, in which R³ group in formula II is a straight chain or branched chain 8 to 11C alkyl group which is optionally substituted by one substituent selected from Br, carboxy including salts thereof, and methoxycarbonyl.

8. A compound according to claim 5, in which R³ group in formula II is a straight chain or branched chain 8-11C alkenyl group, preferably monoethenically unsaturated, which may be substituted by a substituent selected from Br, carboxy including a salt thereof, and methoxycarbonyl.

9. A compound according to any one of claims 1 to 8, in which the groups R¹ and R² together form an oxo group (═O) such that a keto group exists at the C-3 position in the acyl group.

10. A compound according to claim 9, in which the group R³ in formula II will typically be:
(a) an optionally-substituted, saturated or ethylenically-unsaturated, straight or branched chain 8C aliphatic hydrocarbyl group;
(b) a substituted, saturated, straight or branched chain 9C aliphatic hydrocarbyl group;
(c) an optionally-substituted, ethylenically-unsaturated, straight or branched chain 9C aliphatic hydrocarbyl group;
(d) an optionally-substituted, saturated or ethylenically-unsaturated, straight or branched chain 10C aliphatic hydrocarbyl group; or
(e) an optionally-substituted, saturated or ethylenically-unsaturated, straight or branched chain 11C aliphatic hydrocarbyl group, and
in the case where the group R³ is substituted, it is substituted by one or more substituent groups selected from the group consisting of halo, F, Cl, Br or I; 1-6C alkoxy, methoxy, ethoxy, n-propoxy, iso-propoxy, n-butoxy, iso-butoxy and tert-butoxy; carboxy including salts thereof, 1-6C alkoxycarbonyl, methoxycarbonyl, carbamoyl, N,N-dimethylcarbamoyl, and NR⁵R⁶, wherein R⁵ and R⁶ are each selected from H and 1-6C alkyl or R⁵ and R⁶ together with the nitrogen atom to which they are attached form a morpholino group or a piperazino ring, optionally substituted at the 4-N by a methyl group.

11. A compound according to claim 7 or claim 8, in which the substituent is attached in a terminal position on the alkyl group.

12. A compound according to any preceding claim, in which the acyl groups R of formula II in which R³ is a saturated hydrocarbyl group include:
3-oxoundecanoyl;
11-bromo-3-oxoundecanoyl;
10-methyl-3-oxoundecanoyl;
6-methyl-3-oxoundecanoyl;
3-hydroxydodecanoyl;
12-bromo-3-oxododecanoyl;
3-oxotridecanoyl;
13-bromo-3-oxododecanoyl;
3-hydroxytetradecanoyl;
3-oxotetradecanoyl;
14-bromo-3-oxotetradecanoyl; and
13-methoxycarbonyl-3-oxotridecanoyl.

13. A compound according to any preceding claim, in which the acyl groups R of formula II in which R³ is an ethylenically unsaturated hydrocarbyl group include:
3-oxo-12-tridecenoyl;
3-oxo-7-tetradecenoyl;
3-hydroxy-7-tetradecenoyl;
3-oxo-9-tetradecenoyl;
3-hydroxy-9-tetradecenoyl;
3-oxo-10-tetradecenoyl;
3-hydroxy-10-tetradecenoyl;
3-oxo-11-tetradecenoyl;
3-hydroxy-11-tetradecenoyl;
3-oxo-13-tetradecenoyl; and
3-hydroxy-13-tetradecenoyl.

14. Use of a compound according to any one of claims 1 to 13 in a medicament for the modulation of immune response in the animal body.

15. Use of a compound according any one of claims 1 to 13, in which the animal is a mammal.

16. Use of a compound according to any one of claims 1 to 13, in which the mammal is a human.

17. Use of the compound of any one of claims 1 to 13 in the manufacture of a medicament for the inhibition of lymphocyte proliferation.

18. Use of the compound of any one of claims 1 to 13 in the manufacture of a medicament for the down-regulation of TNF-□ secretion by monocytes/macrophages and the consequent activation of Th 1 lymphocytes in humans.

19. A pharmaceutical composition comprising a therapeutically-effective amount of the compound according to any one of claims 1 to 13 or an enantiomer thereof.

20. Use of the compound of any one of claims 1 to 13 including enantiomers thereof, for the manufacture of a medicament for the treatment of a disease of a living animal body including human which disease is responsive to the activity of an immunosuppressant.

21. Use according to claim 20, in which the disease is an autoimmune disease.

22. A method of treating a disease of a living animal body, including a human, which disease is responsive to the activity of an immunosuppressant, e.g., an autoimmune disease, which method comprises administering to the living animal body, including human, a therapeutically-effective amount of a compound according to claim 1.

23. Use of compound according to any one of claims 1 to 13, in which the compound is orally administered.
